(19) United States
(12) Patent Application Publication
(10) Pub. No.: US 2008/0300493 A1
(43) Pub. Date: Dec. 4, 2008

(54) Optical microprobe for blood clot detection

(76) Inventors: Rodolfo Gatto, Chicago, IL (US); Enrico D'amico, Frascati (RM) (IT); William W. Mantulin, Champaign, IL (US); Enrico Gratton, Irvine, CA (US); Fady Charbel, River Forest, IL (US)

Correspondence Address: GREENLEE WINNER AND SULLIVAN P C, 4875 PEARL EAST CIRCLE, SUITE 200, BOULDER, CO, 80301, US

(21) Appl. No.: 12/134,757

(22) Filed: Jun. 6, 2008

Related U.S. Application Data
(63) Continuation-in-part of application No. PCT/US06/61742, filed on Dec. 7, 2006.
(60) Provisional application No. 60/748,289, filed on Dec. 7, 2005.

(51) Int. Cl. A61B 5/02

(52) U.S. Cl. 600/479

(57) ABSTRACT

The invention is devices and related methods for detecting blood clots in a blood vessel. An optical microprobe is configured to illuminate a blood vessel with electromagnetic radiation corresponding to the near-infrared portion of the electromagnetic spectrum. The optical microprobe has a pair of fiber optic strands configured for transmission spectroscopy to obtain the absorption spectrum generated by the components within the blood vessel. Because blood clots generate a detectable and unique spectrum, the presence or absence of the blood clot is determined by examining the blood vessel absorption spectrum. A specially-designed holder is configured to stably position the optical microprobe relative to the blood vessel and is used to facilitate precise blood clot detection along a length of blood vessel.

### CROSS REFERENCE TO RELATED APPLICATIONS

This application is a continuation-in-part of PCT/US2006/061742 filed Dec. 7, 2006 which claims benefit of U.S. Provisional Application No. 60/748,289 filed Dec. 7, 2005, each of which are incorporated herein by reference in their entirety to the extent not inconsistent herewith.

### BACKGROUND OF THE INVENTION

A major complication during vascular surgery is blood clot formation. Blood clots adversely impact blood flow, result in tissue damage related to hypoxia, and are associated with other serious medical conditions such as stroke. The present invention relies on the finding that blood clots in a blood vessel generate a unique and specific spectrum detectable by transmission spectroscopy. The devices and methods of the present invention non-invasively illuminate the blood vessel and by transmission spectroscopy determine efficiently and reliably whether a blood clot is present within the blood vessel. The devices and methods disclosed herein are particularly useful during vascular surgery to detect whether or not blood clots are within a blood vessel.

Optical flow meters that measure blood flow are known in the art. For example, laser Doppler detection schemes or ultrasound-based systems are often used to assess blood flow and assist surgeons in evaluating the hemodynamic status of a blood vessel. The drawback of those systems is that although they may identify a vessel obstruction (e.g., a blood clot), they are unable to localize the position or the extent of the obstruction. Consequently, the surgeon is required to painstakingly backtrack along the vessel to find a region where flow can be detected and then to surgically search in the intervening length of the vessel for the clot. This is often a time-consuming procedure, increasing total surgical time and placing the patient at additional risk. Furthermore, the multiple cuts to the vessel that are often associated with surgically locating a blood clot are not conducive to vascular health.

When blood clots form during neurosurgery, blood flow is often reduced in the vessel containing the clot (depending on the cross-sectional area of the lumen blocked by the clot) and so the brain tissue volume irrigated by that vessel may become hypoxic. If hypoxic conditions persist, temporary or even permanent brain damage may result. In general, blood clot development (and attendant oxygen deprivation, hemodynamic changes, thrown clots, strokes) can occur in other vascular surgical procedures, such as bypass, stent insertion and angioplasty. While flow measuring devices are useful to the surgeons in establishing poor or non-existent blood flow in the surgical field, these devices do not provide any information regarding the actual location of the clot. For that reason, common practice involves sequential upstream surgical incisions into the vessel to locate and excise the clot. The optical microprobe of the present invention rapidly and accurately identifies the location and the extent of the blood clot in the vessel and facilitates timely intervention by the surgeon. Rapid and efficient response to a blood clot minimizes tissue and blood vessel damage and decreases the likelihood of serious complications such as stroke.

The devices and methods presented herein present significant improvements over devices presently used. For example, many imaging devices require the source to be invasively placed within the blood vessel lumen (e.g., U.S. Pat. No. 6,178,346). Such a configuration results in additional surgical effort and damage to the blood vessel. The present invention avoids this drawback by placing both the optical source and the optical detector outside the blood vessel.

Many devices are not configurable for transmission spectroscopy, and instead rely on different configurations such as for epi-illumination to measure reflectance, for example (e.g., U.S. Pat. No. 6,104,939). Devices based on reflective spectroscopic methods (or scattering) are unable to accurately assess absorption changes in the blood vessel that is associated with blood clot formation. Probes known in the art that do not isolate a particular blood vessel (e.g. plethysmographs for oxygen monitoring or large tissue area imagers as in U.S. Pub. No. 2003/0236458) are unable to provide sufficiently detailed and position-specific images within a particular blood vessel to be of use to the vascular surgeon. The present invention overcomes these obstacles by providing a robust and easy-to-use optical microprobe that can rapidly image an entire length of blood vessel for blood clots in a non-invasive manner.

### SUMMARY OF THE INVENTION

The invention uses the finding that a blood clot absorbs electromagnetic radiation (“emr”) in the near infra red (“NIR”) wavelength range differently than the surrounding medium within the blood vessel. The unique absorption spectrum associated with a blood clot is used as the basis for devices and methods that non-invasively and rapidly detect blood clots in a blood vessel, by illuminating the blood vessel with emr and detecting by transmission spectroscopy the presence, absence or magnitude of the absorption spectrum that is associated with a blood clot. The algorithms, methods and devices of the invention can further resolve the measured spectrum into its component parts such as oxyhemoglobin (HbO₂), deoxyhemoglobin (HHb), and blood clot. The device is extremely robust and easy-to-use, permitting rapid imaging of entire lengths of blood vessels, thereby providing information as to the actual location of a blood clot within a blood vessel. This axial-imaging capability reduces the need for a vascular surgeon to undertake exhaustive and time-consuming searches to pinpoint the obstructed region. The device and methods presented herein can be used to detect blood clots that often form during common vascular surgical procedures.

In an embodiment, the invention is an optical microprobe that is capable of non-invasively detecting blood clots in a blood vessel by transmission spectroscopy. “Non-invasively” refers to the microprobe being able to detect clots without having to enter the blood vessel. This is in contrast to many devices known in the art that require the probe be at least partially contained within the blood vessel lumen.

The optical microprobe has an optical source for generating electromagnetic radiation having a range of wavelengths capable of being absorbed by a blood clot. A range of wavelengths “capable of being absorbed by a blood clot” refers to the blood clot absorbing emr in a manner that is different than the other absorptive components (e.g., HHb, HbO₂), thereby permitting blood clot detection by examining the absorptive spectrum. In an aspect, these wavelengths correspond to the wavelength of NIR (e.g., wavelength having a range of between about 600 nm and 1200 nm). A first fiber optic strand capable of transmitting the electromagnetic radiation generated by the optical source is connected to the optical source at a proximal end, and the distal end of the first strand is capable of illuminating the blood vessel, such as illuminating the blood vessel with the range of wavelengths capable of being absorbed by a blood clot. A second fiber optic strand capable of transmitting the emr generated by the optical source, collects the emr transmitted through the blood vessel at a distal end. Optionally, the proximal end is connected to additional devices downstream useful in determining whether a blood clot is present, such as a spectrophotometer, analyzer and/or a display.

To ensure the distal ends of the fiber optic strands are appropriately positioned in a diametrically opposed configuration (e.g., on either side of the blood vessel, with the vessel diameter interposed), a holder is provided having a pair of holding arms, such as a first holding arm connected to the first fiber optic strand distal end, and a second holding arm connected to the second fiber optic strand distal end. By rigidly positioning each of the holding arms connected to the fiber distal ends, the holder is capable of stably positioning the optic strands in a diametrically opposed configuration, and separated by a separation distance. In an aspect, the fiber optic strands are flexible, to permit versatile optical microprobe positioning. The holder ensures that even for flexible fibers, the ends can be stably positioned relative to a blood vessel disposed between the distal fiber ends. In an aspect, the distal ends of the fiber optic strands are separated by a distance equal or slightly greater (such as 5% or greater, 10% or greater or between about 5% and 10% greater) than the outer diameter of the blood vessel (e.g., lumen diameter plus twice the vessel wall thickness). Alternatively, the fiber distal ends can physically contact the outer wall of the blood vessel.

The dimension of the fiber optic light source strand influences the dimension of the emr beam that illuminates the blood vessel. In the simplest embodiment, the distal fiber strand source has a fixed dimension, so that the illumination beam exits the fiber source with a fixed dimension. So long as the emr that illuminates the blood vessel is appropriately positioned (and more specifically the portion of the emr that travels through the blood vessel) to pass through a clot within the blood vessel, and the clot is able to measurably absorb a portion of the emr, the system is capable of detecting the clot. Accordingly, to maximize the likelihood that at least a portion of the source emr is positioned to pass through the clot, a preferable embodiment is for an emr illuminating dimension that is about the diameter of the blood vessel lumen. In an embodiment, the dimension of the light beam exiting the distal end of the first fiber optic strand is about equal to or less than the diameter of the blood vessel lumen. In an embodiment, the dimension of the light beam is less than the diameter to the blood vessel lumen. In an aspect of the invention, lens and other optical control elements such as diffusers, are employed to facilitate control of emr illuminating beam dimension and thereby, the ability to tailor a single optical microprobe of the present invention to a variety of blood vessel sizes, types, and tissue surrounding the blood vessel. In an aspect, the optical microprobe detects blood clots that occupy more than 20%, more than 40%, more than 50%, or more than about 70% of the cross-sectional area of the blood vessel lumen.

In an embodiment, each of the holding arms has a bottom end that is connected to a holding tip. The holding tip connects to the distal end of the first fiber optic strand, and the second holding tip is connected to the distal end of the second fiber optic strand. In an aspect, the distal end of the fiber optic strand is placed within a tip orifice. The orifice has an opening for transmitting emr from the first fiber optic strand that is connected to the optical source to the blood vessel, or for collecting emr that has passed through the blood vessel to distal portion of the second fiber optic strand. The orifice can be a straight passage or a curved passage, with a second opening for receiving a fiber optic strand. The fiber optic strand can be permanently connected in the orifice or can be temporarily connected to facilitate removal of distal fiber optic ends from the tip. The tip is preferentially made of an inert material suitable for contacting blood vessel and tissue, such as a medical-grade plastic.

Any of the optical microprobes of the present invention optionally have means for selecting the separation distance between the distal ends of the fiber optic strands that illuminate the blood vessel with emr and collect transmitted emr. Means for selecting the separation distance is any system known in the art capable of moving one element with respect to another element in a linear fashion and includes, set-screw, micromanipulator, microdrive, computer-controlled positioners. In this aspect, the separation distance means can be connected to each of the holding arms, thereby ensuring the separation means does not interfere with a surgeon's field of view while still providing precise control of separation distance. In an embodiment, the separation distance is selected from the range of about 0.5 mm to about 2 cm, 0.5 mm to 15 mm, or 0.3 mm to 5 mm. In an embodiment, the separation distance is selected to be about equal to the outer diameter of the blood vessel of interest.

Any of the optical microprobes optionally have a micromanipulator connected to the holder for controllable positioning of the distal ends of the fiber optic strands. Micromanipulators are known in the art and provide controllable positioning on the order of the micron scale in one or more dimensions. For less precise applications (e.g., larger diameter vessels), the manipulators provide controllable positioning on the order of millimeters. In an aspect, the micromanipulator provides three-dimensional positioning capability. In an aspect, the micromanipulator is computer-controlled.

In another embodiment, the device has one or more components optically connected to the proximal end of the second fiber optic cable that collects emr transmitted through the blood vessel. In an aspect, the component is an optical detector optically connected to the second fiber optic strand proximal end for detecting the electromagnetic radiation collected by the second fiber optic strand distal end. The detector itself can be a spectrophotometer, including a commercially-available spectrophotometer capable of measuring emr intensity in the NIR wavelength range.

In an aspect, analyzers are provided that are capable of determining the intensity of electromagnetic radiation at a wavelength or wavelength range corresponding to the wavelength absorbed by a blood clot, including a wavelength range selected from between about 600 nm and 1000 nm, 650 nm to 990 nm, or 649 nm to 979 nm. In a specific embodiment, the analyzer determines the spectral contribution due to absorption of the electromagnetic radiation by a spectral component. The spectral component is selected from the group consisting of HHb, HbO₂, scattering, water, noise (e.g., fluctuations in optical source output intensity) and fat. In an embodiment, the spectral contribution is determined by least squares fitting of each of the spectral components to the measured absorption spectrum, for example over a wavelength range of about 650 nm to about 990 nm. Each of the desired blood vessel components such as HHb, HbO₂, scattering, water, noise due to fluctuation in wavelength intensity produced by the emr source) are fit by spectral decomposition, and specifically least square fitting algorithms as known in the art. This is particularly useful as it can help the surgeon decide the impact a detected clot has on oxygenation levels in the blood vessel (and therefore, the oxygen concentration of downstream tissue supplied by the blood vessel). In an aspect, HHb and HbO₂ are the spectral components whose spectral contributions are determined.

To provide automated control of optical microprobe positioning, any of the devices of the present invention have a microdrive assembly operably connected to the holder to provide blood clot location detection along at least an axial portion of the blood vessel. In an aspect, the first and second optic fiber strand distal ends are capable of physical contact with the blood vessel outer wall. The blood vessel can have any diameter, including a diameter selected from the range of 0.5 mm to 2 cm, 0.5 mm to 1 cm, or 0.5 mm to 5 mm. Alternatively, for the aspect where the distal ends are stored within the holding tip, the holding tip is capable of establishing physical contact with the blood vessel outer wall.

In an embodiment, the optical source is a white-light source. In an embodiment, the optical source generates electromagnetic radiation substantially restricted to the near infrared portion of the electromagnetic spectrum. “Substantially restricted” refers to greater than 50%, greater than 70% or greater than 90% of the integrated spectral output falling within at least a portion of the NIR wavelength range.

In another embodiment, the invention is a method for detecting clots in a blood vessel. The method uses any of the devices disclosed herein. In an aspect, the method is providing a first optical fiber having one end in optical contact with the outer surface of the blood vessel and the other end in optical contact with an optical source. A second optical fiber is provided in optical contact with the outer surface of the blood vessel, wherein the first and second optical fibers are positioned in a diametrically-opposed configuration. Electromagnetic radiation is generated by the optical source and used to illuminate the blood vessel with electromagnetic radiation, wherein the emr contains wavelengths that are capable of being absorbed by a blood clot within the blood vessel and at least a portion of the illuminating radiation passes through the blood vessel. The second optical fiber collects at least a portion of the emr that has passed through the blood vessel. The collected emr is detected and a radiation spectrum having a wavelength between about 600 nm and 1000 nm obtained, wherein the spectrum is sensitive to blood clots. The detected radiation is analyzed to determine the presence or absence of a blood clot.

In an embodiment, the analyzing step determines the spectrum over a wavelength range of between 650 nm and 980 nm. The analyzing step can use any means known in the art to determine whether a detected spectrum has a component due to a blood clot. For example, the detected radiation spectrum can be compared to a standard blood clot spectrum, wherein the standard blood clot spectrum is obtained from an in vitro blood clot. The analyzing step optionally determines the fractional contributions due to blood clotting by spectral decomposition. In addition, the analysis step can optionally detect from the spectrum one or more blood parameters selected from oxyhemoglobin, deoxyhemoglobin, total hemoglobin (tHb), and intravascular oxygen saturation (SO₂). In an aspect, the blood clot is detected by measuring the rate of change of the spectral contribution of the one or more spectral components and comparing the measured rate of change or total change to a baseline value. For example, if the absorption coefficient of any of the spectral components such as HHb or HbO₂ is 50%, or 60% greater than the baseline value, a blood clot is considered to be forming or formed. The baseline value is chosen based on the absorption spectrum for unclotted blood or the absorption spectrum in the blood vessel at time zero before any clots have formed.

Any of the methods disclosed herein optionally have an additional step for moving the optical fibers along at least a portion of the blood vessel length to obtain a radiation spectrum along at least a portion of the blood vessel length, thereby determining the location of the blood clot.

Any of the methods and systems provided herein are capable of use in a wide variety of surgical situations, including preoperative, intraoperative and/or postoperative. Preoperative refers to assessment prior to surgery, such as vessel evaluation for surgical intervention related to one or more of: brain aneurysms, ischemic strokes, arteriovenous malformation (AVM), peripheral artery disease, reconstructive plastic surgery, moyamoya disease. Intraoperative includes surgical situations including vascular manipulation and/or transitory vascular clamping where clot development is a concern. Examples where such manipulatons occur are with insertion/removal of vascular stents, bypass anastomoses for revascularization including coronary and carotid bypass surgery, bypass anastomoses for revascularization of peripheral vessels as in the treatment of deep vein thrombosis. Methods and devices presented herein provide for rapid real-time evaluation of clot development, formation and optionally clot localization in a minimally invasive manner, thereby improving patient outcome after the surgical intervention.

In another aspect, a method is provided that uses any of the optical microprobes of the present invention to image a length of blood vessel to determine if a blood clot is present within the imaged length of blood vessel. The optical microprobe is positioned so that a blood vessel is between the distal ends of the fiber optic strands. The blood vessel is illuminated and the absorption spectrum obtained. The process is sequentially repeated along a length (or a portion thereof) of the blood vessel (e.g., different axial positions) to determine the precise axial location of a blood clot, if present. This permits the surgeon to remove the blood clot without having to undertake a painstaking visual search requiring a number of cuts to the blood vessel.

### BRIEF DESCRIPTION OF THE DRAWINGS

FIG. 1 is a schematic illustration of the configuration of an embodiment of the present invention useful for assessing clot formation in a blood vessel. The arrows illustrate the direction that electromagnetic radiation (“emr”) (or information) travels, beginning with an optical source connected to a fiber optic for illuminating the blood vessel. The inset picture is an expanded view illustrating the configuration of the holder and each of the fiber optic source and collector relative to the blood vessel (blood vessel coming out of the page in the inset). The connection between the spectrometer and the laptop or personal computer is either a USB cable (USB spectrometer) or a PC 25 pin cable (PCI interface spectrometer).

FIG. 2 is a photograph of an optical microprobe system that is positioned to image a rat microvessel to detect blood clots within the blood vessel.

FIG. 3 is a flow diagram summarizing the configuration and processes of the invention.

FIG. 4 is an in vitro absorption spectrum obtained by transmission spectroscopy of blood in a cuvette. The smooth curve is the absorption spectrum after the blood has completely clotted (axis on left). The plot shows the spectral absorption of the blood clot. The fluctuations are due to the light source intensity fluctuations during the measurement.

FIG. 5 is a plot of relative absorption as a function of wavelength for whole blood in vitro. The upper line is for the clot and lower line is baseline absorption spectrum for when the blood is initially placed in the cuvette and has not clotted. The fluctuation in light source intensity is compensated to obtain a smooth curve.

FIG. 6 is a plot showing venous spectrum specific components. The plot shows the relative amount of HbO₂ (bottom line) and HHb (deoxyhemoglobin top line) in a venous blood vessel.

FIG. 7 is similar to FIG. 6, except the spectrum specific components for HHb and HbO₂ are obtained from the abdominal aorta and the data are not smoothed.

FIG. 8 is a three-dimensional plot of the spectrum obtained from a blood vessel that has been clamped. The spectrum changes with time as the extent of clot formation increases with time since clamping.

FIG. 9 is a plot showing temporal changes in OxyHb, DeoxyHb and blood clot spectrum during blood vessel clamping. The plots show absorption of each of the components as a function of time since clamping. The blood clot component is obtained by selecting the last spectrum measured in the clamping experiment (corresponding to blood clot as verified by visual examination) which shows the clot absorption spectra signature; the other components are fit to the spectrum. The blood clot, HHb and HbO₂ absorption are fit over a range of about 649 nm to about 979 nm.

FIG. 10 summarizes the basic spectral components (not to scale) that can be used in fitting algorithms that determine spectral contributions of components HbO₂, HHb, scattering, water and/or fat to a measured absorption spectrum. For example, one or more of the spectral components (and preferably at least HHb, and HbO₂) are fit to the measured blood vessel absorption spectrum, thereby determining the spectral contribution of the one or more spectral components. Not shown, is a spectral component that compensates for fluctuations in intensity of light source emr output. In the blood clot detection methodology of the present invention, fat is not used as a component.

FIG. 11 Time series—NIR spectral changes of arterial blood during the coagulation process.

FIG. 12 Time series—NIR spectral changes of heparinized arterial blood during the coagulation process.

FIG. 13 Time series—NIR spectral changes of venous blood during the coagulation process.

FIG. 14 Time series—NIR spectral changes of heparinized venous blood during the coagulation process.

FIG. 15 Absorption changes in arterial blood during the coagulation process between different NIR wavelengths n=1.

FIG. 16 830 nm wavelength changes in arterial and venous 3blood during the coagulation process n=5.

FIG. 17 830 nm wavelength changes in arterial and 5heparinized blood during the coagulation process. n=3.

FIG. 18 830 nm wavelength changes in venous and venous heparinized blood during the coagulation process n=5.

### DETAILED DESCRIPTION OF THE INVENTION

Referring to the drawings, like numerals indicate like elements and the same number appearing in more than one drawing refers to the same element. In addition, hereinafter, the following definitions apply:

The term “electromagnetic radiation” (“emr”) refers to waves of electric and magnetic fields. Electromagnetic radiation useful for the methods of the present invention includes, but is not limited to, light, infrared light, and more specifically near infra-red light (“NIR”).

“NIR” is used herein to refer to light having a wavelength between about 600 nm and 1200 nm, and all subranges encompassed by that range. NIR refers to, for example, a wavelength selected from the range of 640 nm to 1000 nm, 649 nm to 979 nm, or about 660 nm to about 990 nm.

“Blood vessel” is used to refer to any vessel in which information about blood clotting is desired. The term refers to both feeding blood vessels (e.g., arteries) and collecting blood vessels (e.g., veins). The methods and devices of the present invention can be configured to image any diameter blood vessel in an animal or human, for example blood vessels having a diameter range of between about 0.5 mm to 2 cm, 0.5 mm to 1 cm or about 0.5 mm to about 5 mm.

“Fiber optic” is used broadly to refer to cables capable of guiding light without unduly affecting the spectrum. Suitable fiber optics are well known in the art and are commercially available (e.g., Schott Inc., Elmsford, N.Y.). The fiber optics can be flexible or rigid, as needed.

“Optical contact” or “optically connected” refers to one element that generates or transmits emr being capable of illuminating another element. For example, the term encompasses an emr source and fiber optic connected in such a manner that the emr produced by the source is transmitted by the fiber optic strand. The term also encompasses the distal end of a fiber optic source strand that illuminates a blood vessel, as well as the distal end of the collector fiber optic strand that receives emr that has passed through the blood vessel. In such a system, each of the fiber optic strands is said to be optically coupled or connected to the blood vessel. Although an optically connected blood vessel and optic strand can be in physical contact, the term encompasses a strand that does not physically contact the blood vessel.

“Illuminating” refers to emr that leaves the fiber optic and optically contacts the blood vessel. The emr can be a focused beam that passes through the center of the blood vessel, or a beam that passes through a substantial portion of the blood vessel, such as more than 50%, more than 70%, more than 90%, or about the entire cross-section of the blood vessel lumen. “Collecting” refers to capturing substantially all the emr that has passed through the blood vessel and preserving it for transmission spectroscopy detection and analysis to determine whether there is a blood clot contribution to the collected emr.

“Spectral component” refers to an element that is being fitted to the measured absorption blood vessel absorption and includes biological components within the blood vessel capable of absorbing NIR used to illuminate the blood vessel (e.g., HHb, HbO₂, water, fat) and physical factors (e.g., scattering, noise such as fluctuations in optical source output intensity). As known in the art, a curve can be fit to one or more spectral components, such as by least squares fitting, thereby determining the “spectral contribution” of each spectral component used in the fit. In an aspect, clot development or detection is determined by analyzing the one or more spectral contributions from the one or more spectral components.

“Axial portion” of a blood vessel refers to a longitudinal segment of a blood vessel, including substantially the entire length of blood vessel within the surgical field of view.

One of the major complications during vascular surgery is blood clot formation. Disclosed herein is a technique to determine the presence, development and extent of blood clots using a surgical microprobe to illuminate a blood vessel with light having at least a portion with a wavelength corresponding to the near infrared. In vitro near infrared spectrometry characterization on blood clots confirms that clots can be detected with the devices and methods of the present invention. The technique is used on a blood vessel in vivo to show the blood clot signature spectrum and its temporary growth during clamping tests. The light is sent and recovered through flexible optical fibers in contact with the wall of the vessels. To obtain blood clot spectrum signature, the detected in vitro spectrum is compared and fitted with the in vivo collected data, thereby calibrating the in vivo spectrum to permit in vivo detection of clot formation. Alternatively, where blood clots are detected in vivo without relying on a corresponding in vitro blood clot spectrum, the relative changes in concentration of the oxy and deoxy hemoglobin components of the actual spectrum are tracked over time. The clot spectrum contains a higher contribution of HHb compared to HbO₂; by monitoring the relative increase of these components during the experiment or procedure, the curve-fitting procedure can detect when the clot is occurring and its stage of development. Relative concentration of oxyhemoglobin, deoxyhemoglobin and the blood spectrum components are measured in real-time. The presence of the induced blood clot is confirmed by dissection and direct visual inspection of the vessel after the test is completed. The optical probe and associated system is able to characterize spectroscopically the physiological changes that occurs during the period of blood clot formation. This optical system is non-invasive and is able to isolate and track blood clot location inside vessels.

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given.

### EXAMPLE 1

### Optical System for Detecting Clots

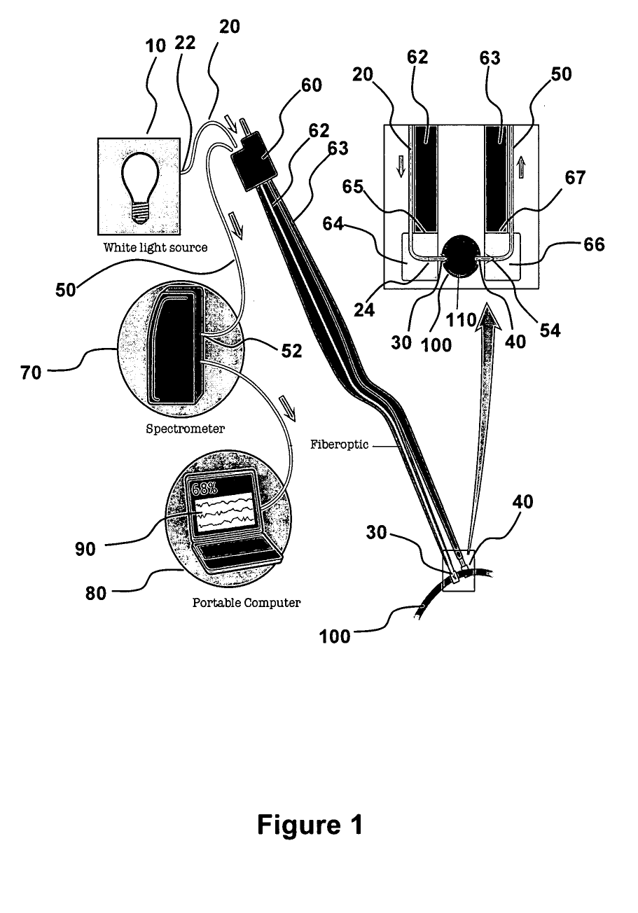

Referring to FIG. 1, electromagnetic radiation (“emr”) source 10 is connected to first fiber optic strand 20 at proximal end 22 and positioned to illuminate blood vessel 100 with emr 30 from distal end 24. A second fiber optic strand 50 collects emr 40 at distal end 54 that has traveled through blood vessel 100. The collector fiber optic 50 transmits the collected emr 40 to a detector 70 that is optically connected at second strand proximal end 52. The detector 70 is illustrated as a spectrometer that is able to measure an optical property of the collected emr 40. In an aspect, the optical property is the intensity of emr 40 (or a parameter obtained therefrom, such as absorbance, relative absorbance, or absorption coefficient) at one or more wavelengths. In an aspect, the optical property is assessed over a wavelength range, such as a spectrum of intensity or absorbance, including a wavelength range that spans all, or a portion of the NIR. The system can be configured to optionally assess light scattering and/or reflection.

The detector 70 is connected to an analyzer 80 that analyzes the detected emr 50 provided by detector 70, to determine whether or not a clot 110 is present within blood vessel 100. Means for displaying 90 the result generated by analyzer is provided so that the outcome of the analysis is conveniently communicated. Means for displaying includes by a video monitor, computer screen, printer, or any other system capable of communicating an outcome to a person. The outcome can be one or more of a spectrum and/or a score indicating the magnitude of the clot.

The inset in FIG. 1 provides a close-up view of the tip of the optical microprobe that illuminates the blood vessel 100 with emr 30 that has been transmitted from the light source to the blood vessel by fiber optic 20 and exits fiber optic strand at distal end 24. On the opposite side of the blood vessel 100, relative to source fiber optic 20 is collecting fiber optic strand 50 that collects emr 40 at distal collecting end 54 that has traversed or passed through the blood vessel 100, and specifically through a blood clot 110. This positioning of optic fiber source and collector on opposite sides of the exterior of the blood vessel is referred to as “diametrically opposed” and ensures that at least a portion of the illumination beam 30 is centered on the vessel and traverses the entire diameter of the blood vessel lumen. To ensure appropriate positioning of fiber optics 20 and 50 (and specifically distal ends 24 and 54 responsible for optical transillumination of blood vessel 100), a holder mechanism 60 provides stable and positionable holding of fibers 20 and 50. In the exemplified embodiment, holder 60 further comprises a pair of holding arms 62 and 63, with holding arm 62 connected to emr source fiber optic 20 and holding arm 63 connected to emr collector fiber optic 50. The fiber optics 20 and 50 are connected to the holding arms 62 and 63 by any means known in the art. The fibers can be integral components of the holding arms by, for example, being disposed within a hollow passage that extends at least an axial portion of holding arms 62 and 63. Alternatively, fasteners may be used to fasten the fiber optic to a surface of the holding arm. Holder 60 is connected to a stand or positioner (not shown) so that optical microprobe is stably positioned.

Additional system utility and flexibility is obtained by connecting special tips 64 and 66 to the ends 65 and 67 of holding arms 62 and 63, respectively, as illustrated in FIG. 1. Fiber optics 20 and 50 can be held by the tips, including by being deposited within orifices spanning tip body 64 and 66, respectively. By constructing the tip of a non-reactive and inert material such as a medical-grade plastic and inserting the fiber optic within the tip body, damage to the blood vessel or surrounding tissue can be minimized. In addition, the fiber optic tip can be kept free of debris that could potentially obstruct emr transmission and/or collection. Instead of the fiber optic directly contacting the blood vessel or surrounding tissue, the tip is optionally the component that makes physical contact, wherein the tip has an orifice for transmitting light to or from the optical fiber. Such a configuration can also facilitate axial tracking of the optical microprobe over the length of the blood vessel.

Holder 60 can further comprise means for positioning tips 64 and 66 such as by one or more stands, manipulators, micromanipulators, actuators, servomechanism, microdrive assembly and electric drive for positioning one or both of holding arms 62 and 64. The positioning mechanism can also include a means for controlling the separation distance between the optical faces of fiber optics 30 and 50 or the distance between opposing faces of tips 64 and 66. The distance between these two faces is preferably greater than or equal to the outer diameter of the blood vessel that is being imaged by the optical microprobe. An optical microprobe that has the ability to vary the distance between the fiber optic probes can be readily configured to test blood vessels of various diameters with maximum sensitivity. Positioning tips 64 and 66 also facilitate axial movement of optical microprobe, thereby providing the capability to easily and rapidly image different axial positions of the blood vessel to better pinpoint axial location of a blood clot. Whereas a fiber optic may get caught, stuck or have an edge that could damage the vessel or tissue, the tip can be of a soft an inert material such as plastic that is smoothably-shaped and better able to move along the outer wall of the blood vessel without causing damage. To minimize the impact on surgical field of view, any of these positioners can be connected to the upper portion of holding arms 62 and 63, or generally in the location of holder 60.

To facilitate cleaning and sterilization for an optical microprobe that is at least partially reuseable, any one or more component parts of the optical microprobe are releasably connected. For example, tips 64 and 66 can be releasably connected to holding arms 62 and 63, to facilitate better sterilization of holding arms 64 and 66. Tips 64 and 66 can be reversibly detached from fiber optic 20 and 50 and discarded, with fresh tips used, or the tips can be sterilized and reused. Fiber optic 20 and 50 have an optional connection that connects an upstream fiber to a downstream fiber, to facilitate more stringent sterilization or disposal of downstream component sections that may have intimate contact with blood vessel or a surrounding tissue, and an upstream portion that does not directly contact the patient. For example, the tip 64 or 66 may be disconnected along with an adjacent section of fiber optic, and sterilized or disposed.

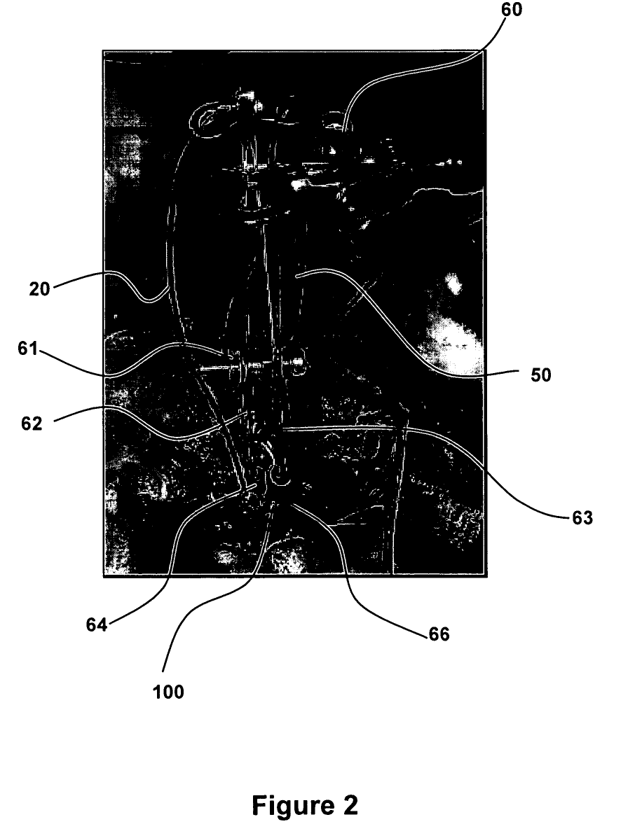

FIG. 2 is a photograph of an optical microprobe prototype used for assessing clot formation in blood vessels. FIG. 2 shows source fiber optic strand 20 connected to a plastic tip 64, wherein the plastic tip is attached to a handle arm 62. Similarly, collector fiber optic strand 50 is connected to plastic tip 66, and the plastic tip is attached to handle arm 63. Between opposing fibers 20 and 50 (e.g., between opposing faces of tips 64 and 66) is blood vessel 100. To appropriately position fibers 20 and 50, and correspondingly appropriately position tips, an optical positioning assembly 61 is positioningly engaged to one or more of holding arms 62 and 63. The embodiment shown in FIG. 2 uses a simple set-screw mechanism 61, wherein the holding arms 62 and 63 are from a single pair of forceps, and the nut positions on the set-screw 61 position arms 62 and/or 63, thereby controlling the separation distance between arms 62 and 63, to allow microprobe separation distance to be tailored to blood vessel diameter, with attendant improvement in signal.

More accurate and precise positioners, holders, and holding arms can be used. For example, holding arms can be connected to a micromanipulator that controls the position of the holder 60, and therefore controlling the position and/or distance separating the holding arms 62 and 63, while ensuring that the illuminating fiber 20 and collecting fiber 50 faces remain aligned on opposite edges of the blood vessel outer wall. A three-dimensional micromanipulator can be attached to the holding arm assembly, to permit fine placement of the entire microprobe assembly. In this manner, the optical microprobe may be precisely positioned such that each of source fiber optic 20 and collecting fiber optic 50 are touching the outer surface, on either side of any size blood vessel. Finally, the entire optical microprobe may be moved along the axial direction of the blood vessel to provide information for precisely pinpointing blood clot location. The three-dimensional micromanipulator can be used to axially position the optical microprobe. Alternatively, a separate actuator, micromanipulator, or servomechanism can provide the axial-positioning means. Any of the micromanipulators can be hand-positioned or connected to a computer-controlled drive for precise positioning of fiber optic distal ends 24 and 54.

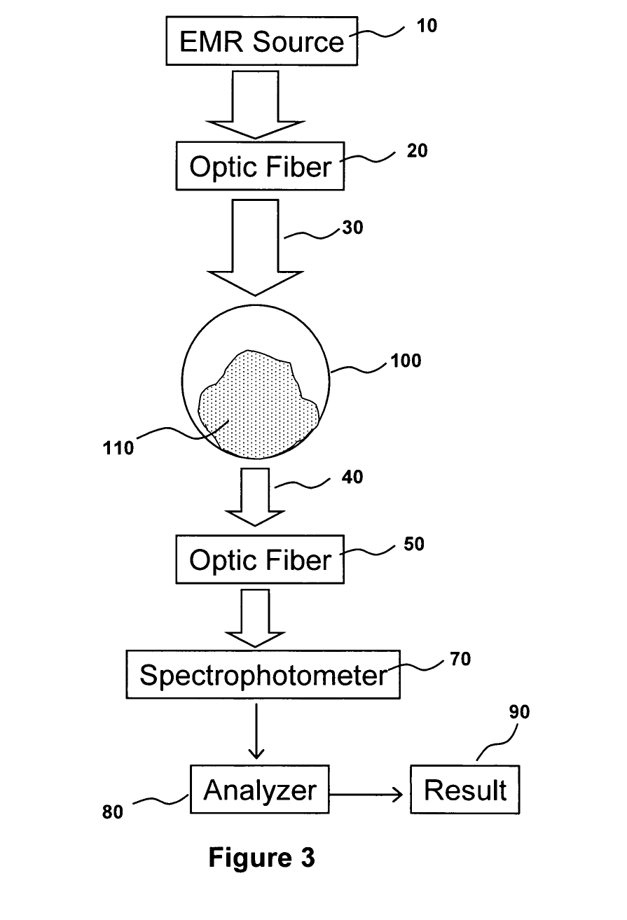

FIG. 3 provides a flow-chart summary of the optical microprobe device and methodology for non-invasively imaging blood-clots within a blood vessel. Source optic fiber strand 20 is optically connected to emr source 10. As indicated by the experimental data provided in the other Examples 2-3, it is important that EMR source emits at least some radiation having a wavelength corresponding to the wavelength of NIR (e.g., 600 nm-1200 nm, 600 nm to 1000 nm, 600 nm-800 nm, or about 650 nm-780 nm), and more particularly a wavelength at which blood clots absorb to a greater extent than other spectral components such as HbO₂ (oxyhemoglobin) and HHb (deoxyhemoglobin). If emr of this wavelength is not generated by the emr source, clots cannot be detected. Accordingly, the emr source can be single-wavelength light source (e.g., laser diode), or a combination of broad or narrow emr source and filters to proved narrow-band emr, so long as the wavelength is one in which a blood clot can absorb. The illuminating emr is broad-band or narrow-band, to generate spectrums suitable for spectral analysis and decomposition methods known in the art. The emr source is optionally a source that produces NIR radiation, including emr substantially restricted to NIR wavelengths. The emr source can be a broadband white-light producer, so long as some of the produced radiation falls within the NIR region.

The source optic fiber illuminates a blood vessel 100 with illuminating emr 30 with at least a portion of the emr 30 having a wavelength capable of being absorbed by a blood clot 110. The blood clot 110 absorbs a portion of the emr 30 that passes through the vessel and clot, as visually depicted by the smaller size of collected emr 40, compared to source emr 30. Collecting optic fiber 50 transmits collected emr 40 to a detector 70 that converts the collected emr 40 into an analyzable spectrum. An analyzer analyzes the spectrum to determine whether a clot is present, and optionally the extent of clot formation. Extent of clot formation is determined by measuring the magnitude of the absorption at clot-specific wavelengths. In an aspect, the longitudinal dimension of a clot is determined by having the optical microprobe scan the blood vessel along the blood vessel length, thereby providing information regarding the length of the blood clot. Information regarding extent of clot formation can be determined by monitoring the relative increase of HHb and HbO₂ absorption at different times. The higher the relative contribution of the HHb component to the actual spectra, the greater the extent of clot formation. Alternatively, if the blood clot spectrum is already known, the contribution of the blood clot component to the total spectra is determined and compared to a threshold value. Any of the information collected and/or analyzed by the optical program can be conveyed to medical personal by a display 90 to take further action (e.g., removing or breaking the clot) as needed. In any of the devices or methods presented herein, means for outputting and/or displaying the result is provided.

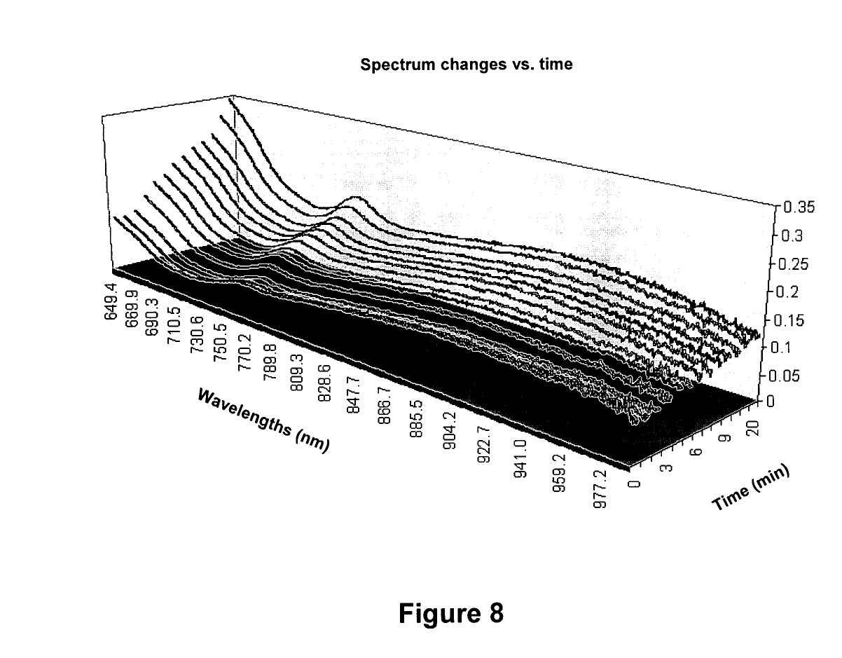

The signal acquired by a spectrometer is processed, analyzed and displayed in computer software developed by the Laboratory of Fluorescence and Dynamics at Urbana Champaign. The software performs a “least square analysis” to minimize the chi square coefficient to obtain the best fit of the measured spectrum. The components for the analysis are chosen depending on the nature of the sample. In the present experiments, HbO₂, HHb, scattering and water. From FIG. 8, the first 30-40 nm of the spectrum (650 nm-690 nm) presents a dramatic increase. Considering an average time of 5 minutes before a blood clot is formed, one definition of blood clot formation is when the absorption coefficient of any of these wavelengths is 50-60% higher than the baseline values.

As the experimental data in the following examples show, any one of a number of methodologies can be employed by one of ordinary skill in the art, to yield information regarding clot formation based on the collected spectrum. Methodologies include multivariate analysis, spectral decomposition, curve fitting, least square fitting of multiple components each having a unique spectrum (e.g., water, HHb, HbO₂, scattering, blood clot and/or noise), and spectral analysis including frequency domain modeling to examine the change in intensity at a particular wavelength that a component is known to absorb. The device is amenable to simultaneously providing spectral information for other variables including HHb, HbO₂, O₂ level, hematocrit, total hemoglobin. This analysis can be conducted using hardware and/or software. For example, software can be employed that incorporates a method known in the art, such as least square curve fitting of multiple components (e.g., such as Elantest™ Software developed by Dr. Gratton, see, e.g., Tanner et al. “Spectrally resolved neurophotonics: a case report of hemodynamics and vascular components in the mammalian brain.” Journal of Biomedical Optics (November/December 2005) 10(6):64009).

### EXAMPLE 2

### In Vitro Clot Detection

Whole blood from the animal is collected and deposited in a cuvette to generate a blood clot. The device pictured in FIGS. 1 and 2 is used to obtain spectral information during the clotting process. Once the blood is completely clotted inside the cuvette, spectral analysis with a spectrometer characterizes and stores the spectral components. Spectral components, with respect to the spectrum over the NIR, refers to the influence of RBCs, and specifically Hb that is either oxygen bound or oxygen unbound, and clot components. Without wishing to be constrained to any particular theory, because the main component of the blood clot is trapped red blood cells that are unable to re-oxygenate, the blood clot spectrum is believed to be similar to the spectrum of HHb.

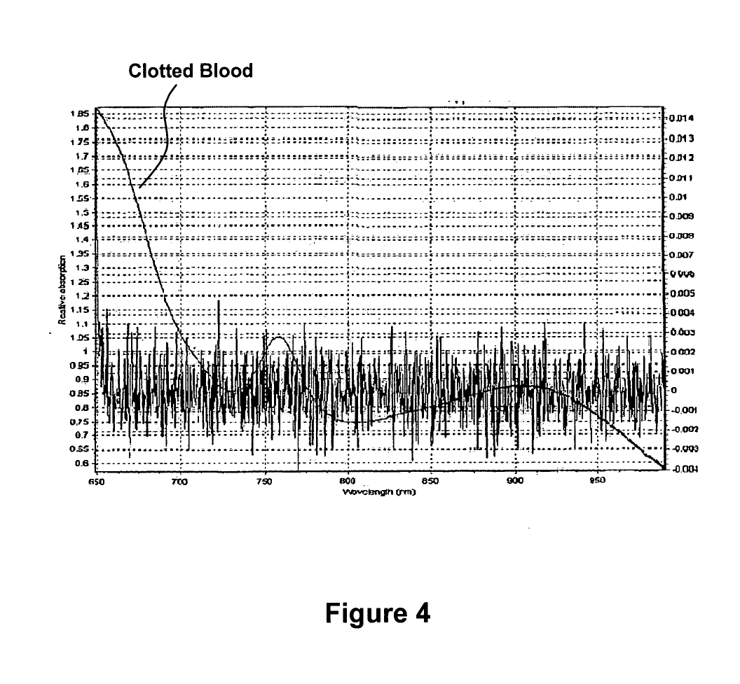

FIG. 4 provides the absorption spectrum of clotted blood in a cuvette. The rapidly fluctuating spectrum is the raw intensity data of the blood. The smooth curve is the relative absorption spectrum of clotted blood. Relative absorption is obtained by comparing the light intensity measured by the detector 70 with and without blood sample in the cuvette. In an aspect, the clotted blood absorption spectrum is used over the displayed range (e.g., about 650 nm to about 970 nm) to assess blood clot formation in vivo. The absorption spectrum of the clotted blood is a linear combination of different components, with higher contributions from the HHb and HbO₂ components. By comparing the spectrum to unclotted blood, it is possible to determine whether a blood clot is present by examining the absorption spectrum over this range. This plot indicates that in the case of blood clots, the HHb component is much higher than for unclotted blood.

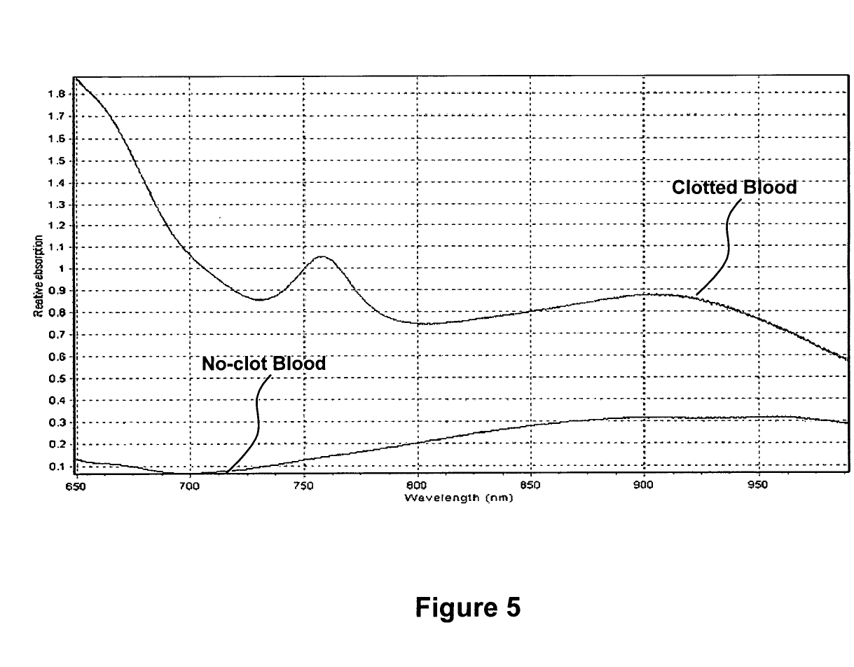

FIG. 5 is a plot of relative absorption as a function of wavelength for whole blood in vitro, similar to that shown in FIG. 4. The upper line is the spectrum obtained after the blood coagulated to form a clot (labeled “blood clot”). The lower line is the absorption spectrum obtained immediately after the blood is introduced into the cuvette and has not yet clotted.

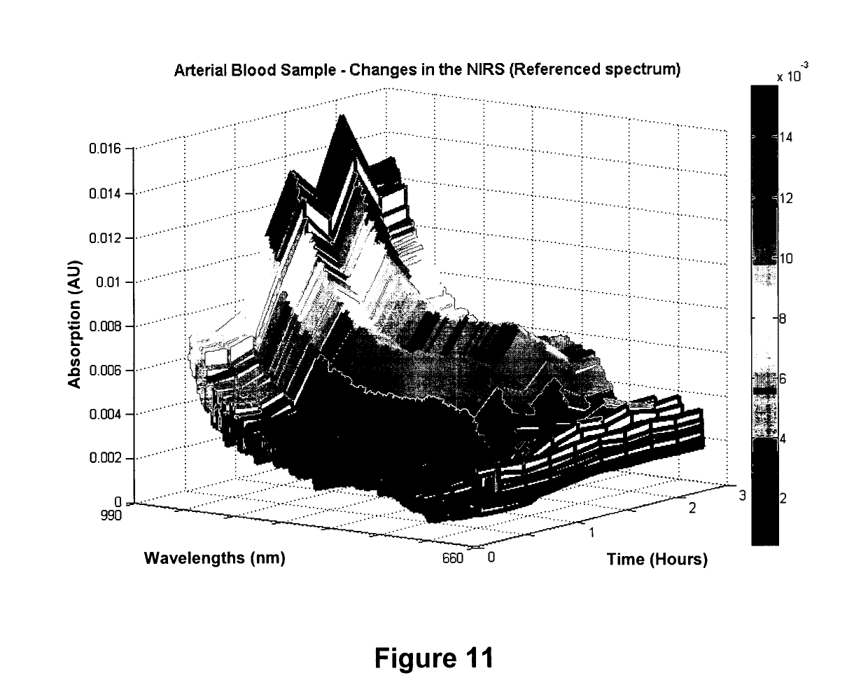

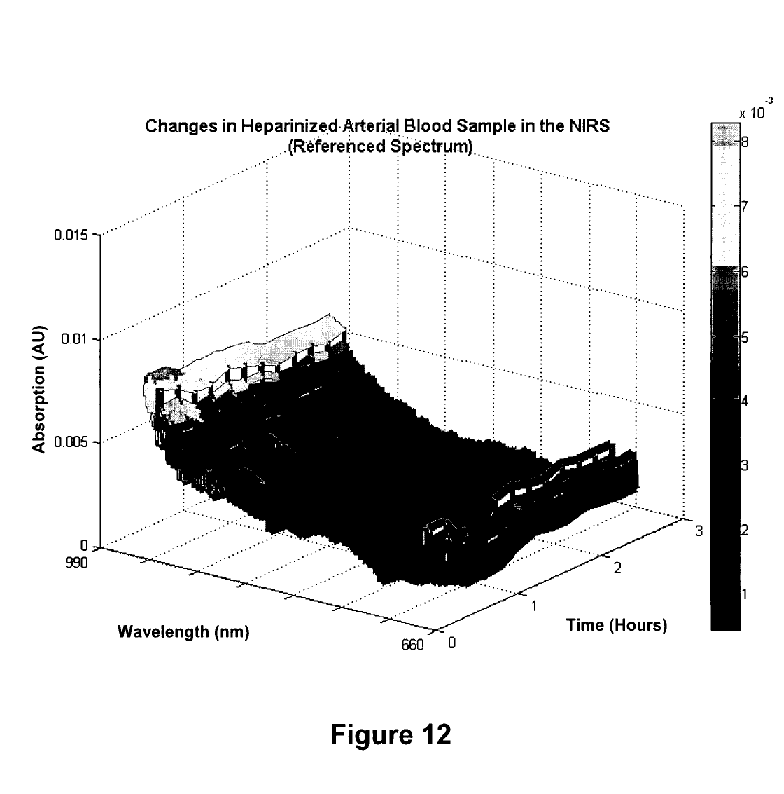

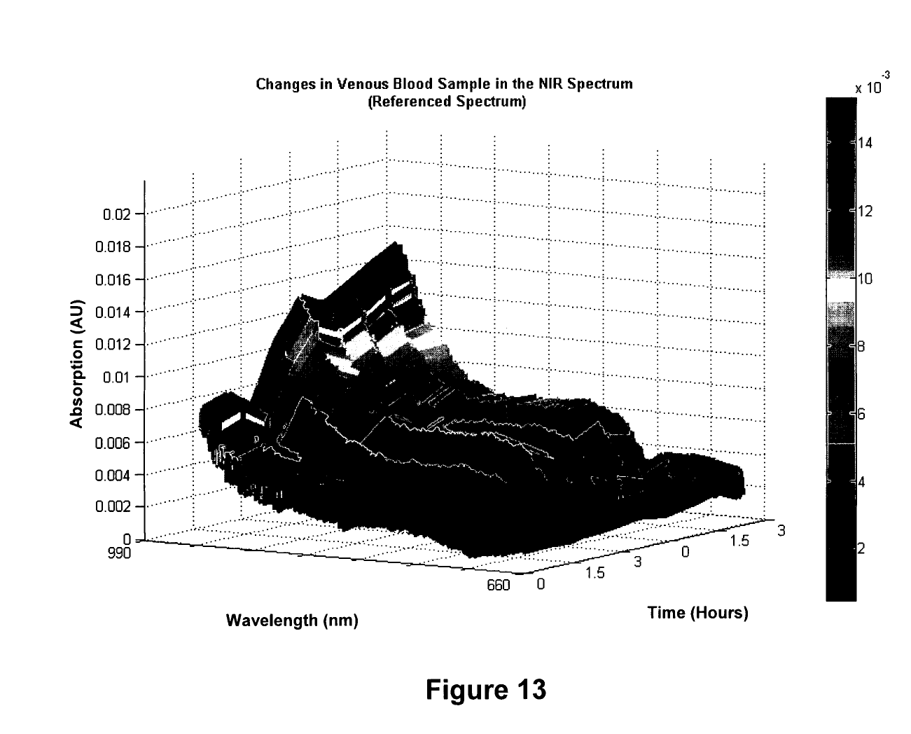

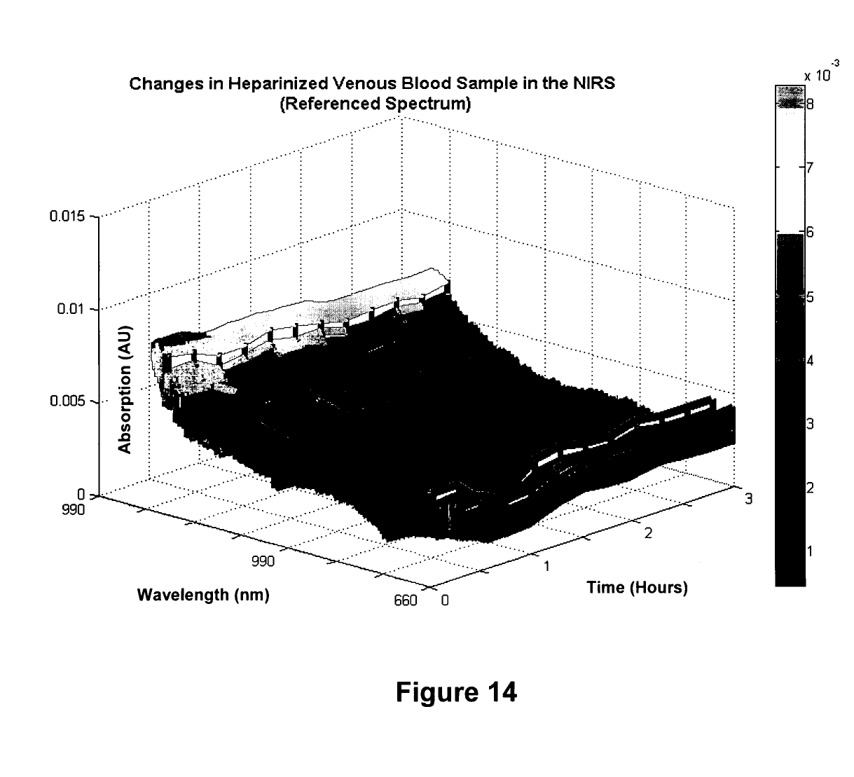

Additional NIR spectra for arterial and venous blood (without and with an anticoagulant) are provided in FIGS. 11-14. There is a detectable change in absorption as early as 40 minutes (FIGS. 11 (arterial blood) and 13 (venous blood)) for the untreated blood. When heparin is added to the blood, the change in spectra is reduced (FIGS. 12 (arterial blood) and 14 (venous blood).

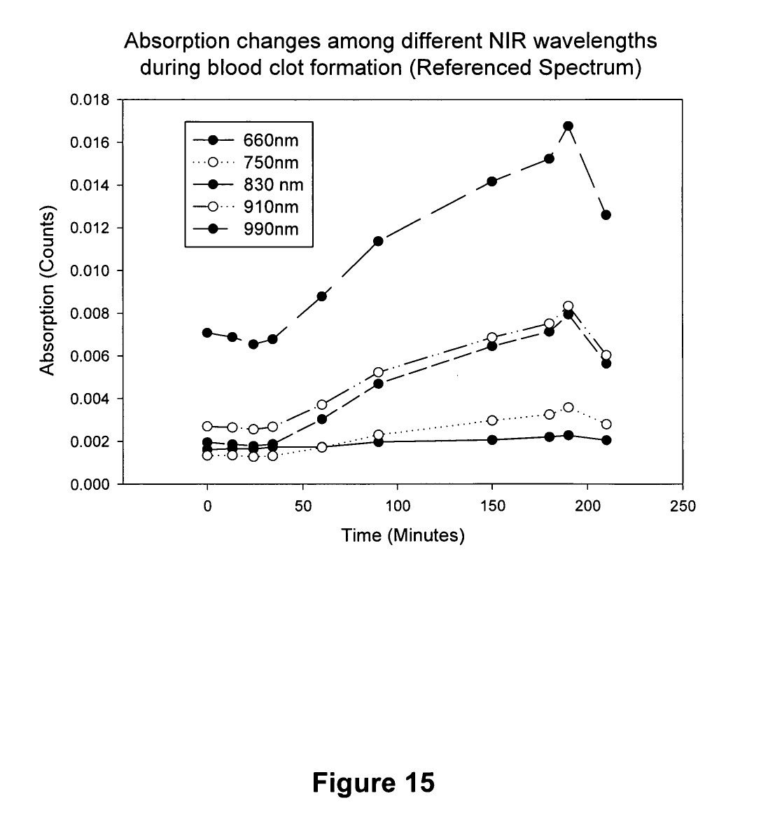

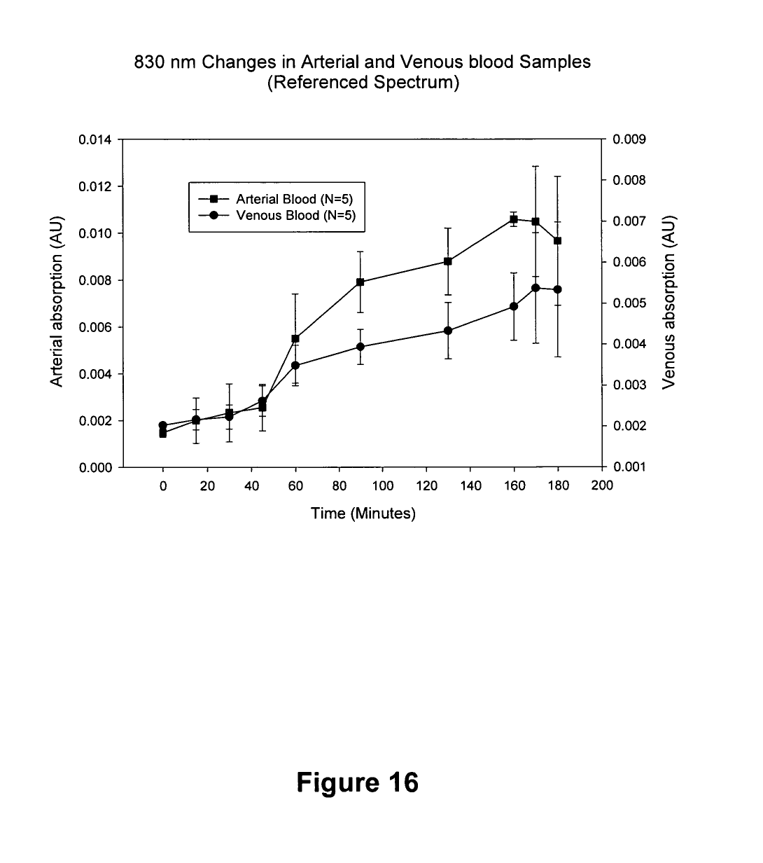

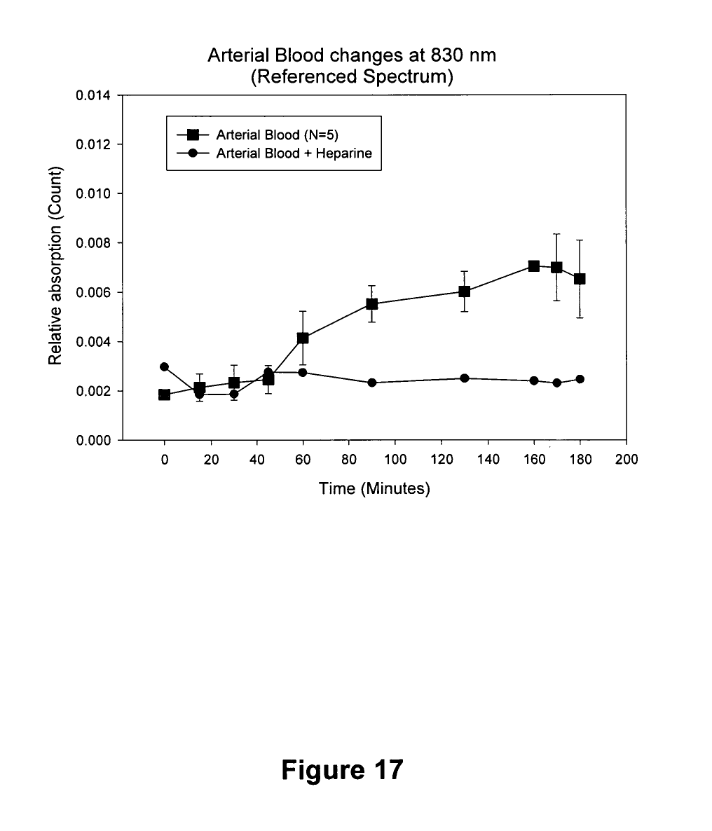

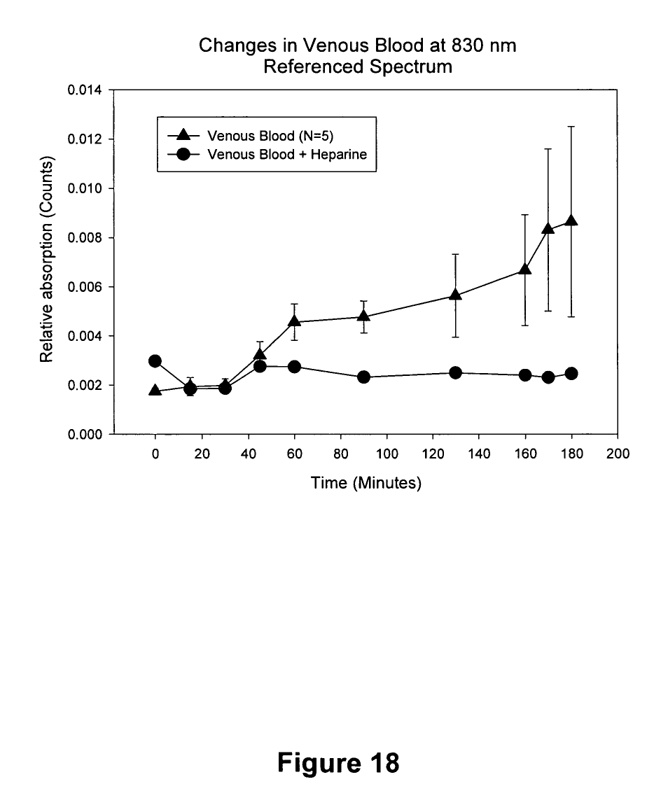

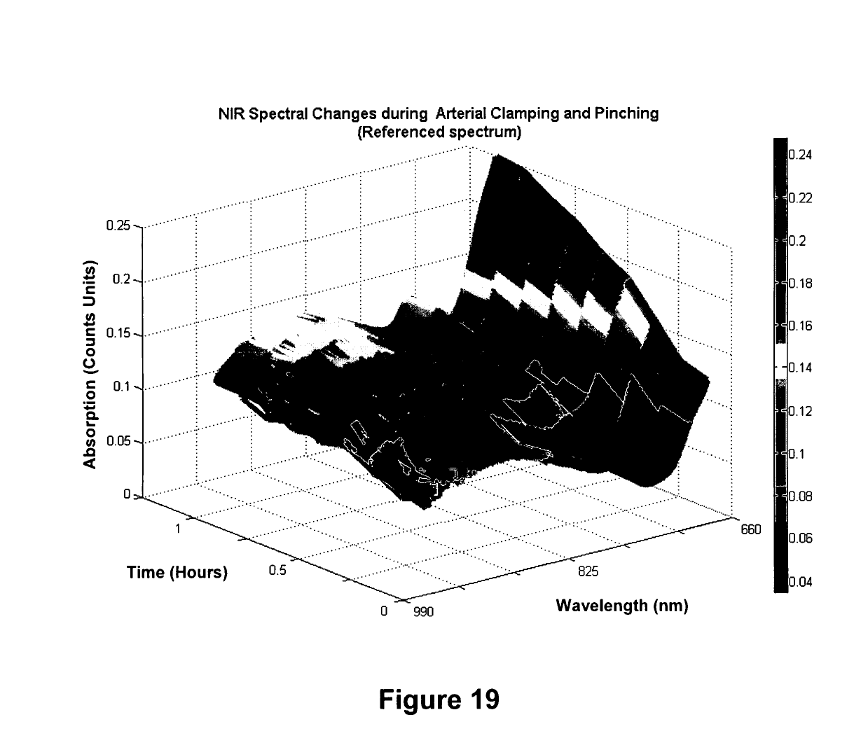

Change in absorption over time for different wavelengths is shown in FIG. 15. Further analysis at a wavelength of 830 nm is provided in FIGS. 16-18, because that wavelength is close to the natural absorption range of oxyhemoglobin. No statistically significant difference between arterial and venous blood samples is detected (see FIG. 16). FIGS. 17-18 show the difference in absorption characteristics at 830 nm when heparin is added to arterial and venous blood, respectively.

The in vitro spectral results provide a starting basis for in vivo spectral analysis. For example, when a clot is located in a blood vessel, an absorbance spectrum change is expected between 600 nm and 1000 nm, including about 650 nm and 1000 nm, that is attributed to absorbance by the blood clot. Assessing the intensity at this wavelength range permits an assessment as to whether (and the extent if any) a blood clot is forming. Using the device summarized in Example 1 also provides the ability to axially locate (within the range of one to a few millimeters) the clot in the blood vessel.

### EXAMPLE 3

### Clot Detection in Blood Vessels

The optical microprobe system used for the in vitro experiments is used on ten six-month old male rats (Rattus Norvergicus Wistard) weighing about 500 g. During the procedure the animals are anesthetized with Ketamine (100 mg/kg), Xylazine (5 mg/kg) and Acepromazine (1.0 mg/kg). Depth of anesthesia is tested by foot pinch every 15 minutes. Supplemental doses of Ketamine 30 mg/kg and Xylasaline 1.75 mg/kg are given as necessary. In order to ensure adequate ventilation and oxygenation of the tissues, a traqueostomy is performed that connects the airway to a ventilator. The ventilator is set by visually observing the degree of lung expansion. The average breath per minute is about 85, producing an average tidal volume of 1.5 mL. The minute volume is 100 mL/min (range 75-130 mL/min). Because this technique is highly sensible to changes in hematocrit and the surgery can result in animal bleeding, measurements of capillary hematocrit and hemoglobin concentration (Hematocrit point H2, Stanbio, Tex.) are performed to evaluate blood loss.

The animal is placed in the surgical field. It is shaved and prepared with Betadine, and a midline incision made from the sternum to the pelvis. The peritoneal organs are retracted to one side and the abdominal vessels isolated. Under a surgical microscope, the abdominal aortic artery and inferior vena cava vein are dissected using standard microsurgical techniques. Once the artery is prepared, the optical microprobe of the present invention is placed in optical contact (or physical contact) with the outer surface of the blood vessel. No physical damage or undue stress need be placed on the blood vessel in order to establish sufficient optical contact. A preferred optical contact configuration is placing each the two fiber optic strands in a location that is diametrically opposed, with the entire diameter of the blood vessel disposed between the facing fiber strands. To ensure the diametrically opposed positioning is stable, each of the fiber optic strands are connected to rigidly positioned tips connected to a holding arm. The optical microprobe instrument configuration isolates the vessel, thereby focusing the light beam directly through the vessel with minimal light source interference.

After probe placement, blood clots are induced in the blood vessel by microsurgical clamping. Short and long clamping protocols are consecutively made in order to avoid the wash out effect due to the simultaneous proximal-distal unclamping, as summarized in TABLE 1. The detected spectrum is fitted with each of the components responsible for the absorption spectrum (e.g., HHb and HbO2) and blood clot is detected by, for example, analyzing the change in the ratio of HHb to HbO₂ over time, for example.

TABLE 1
Clamping Protocol

| Abdominal Aortic | Inferior Cava |
|---|---|
| Artery Clamping | Venous Clamping |
| Baseline-1 minute | Baseline-1 minute |
| Proximal and distal | Proximal and distal |
| clamping-30 minutes | clamping-30 minutes |
| Distal unclamping-5 minutes | Proximal unclamping-5 minutes |
| Proximal unclamping-5 minutes | Distal unclamping-5 minutes |
| (Total unclamping) | (Total unclamping) |

During the tests, the relative amount of oxyhemoglobin (HbO₂) and deoxyhemoglobin (HHb) are correlated with the specific vessel involved.

One example of a time course of spectra change in a blood vessel is provided in FIG. 19. In this example, the vessel is clamped to induce clotting. FIG. 19 illustrates that significant changes in the relative absorption patterns are observed in the NIRS.

The in vitro and in vivo tests indicate that blood clots have a unique absorption spectrum (FIGS. 4-5), which allows for clot identification in vivo. The optical microprobe system and related methods allow tracking the growth of the clot over time and also localization of the spatial dimensions of the clot in the vessel (to a resolution of a few millimeters). We have excellent temporal resolution for data acquisition, ranging from 500 spectra/second to a total acquisition time of minutes to hours. We have tested the concept with an animal model (rat) and we have assembled a prototype device (FIG. 3) that detects blood clots in a blood vessel or in vitro.

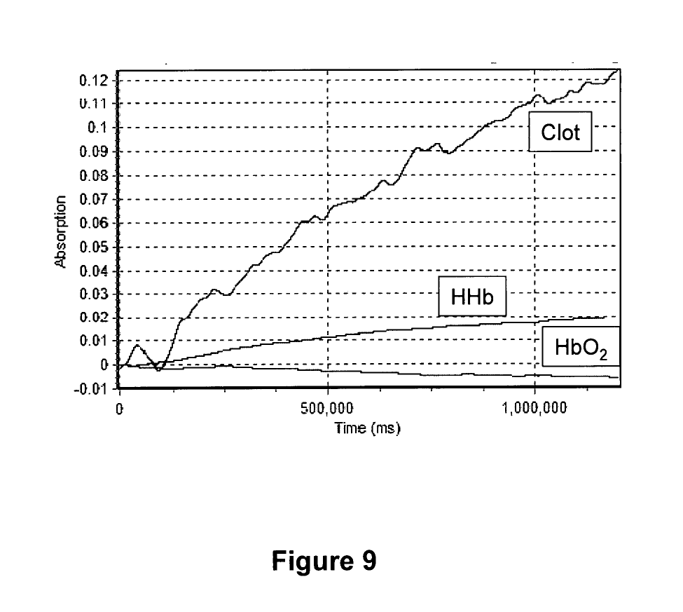

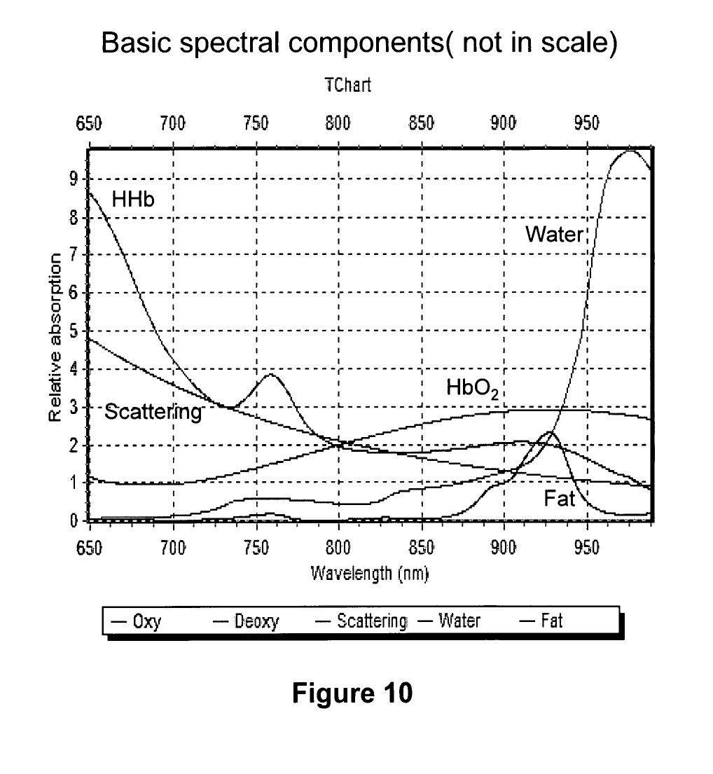

The spectral acquisition hardware is robust. The emr spectrum collected by the fiber optic collector and detected by the spectrophotometer is analyzed, for example, by software that permits the real time detection of relative concentration of oxyhemoglobin, deoxyhemoglobin and blood clot spectrum fractions during vascular clamping procedures (FIG. 9).

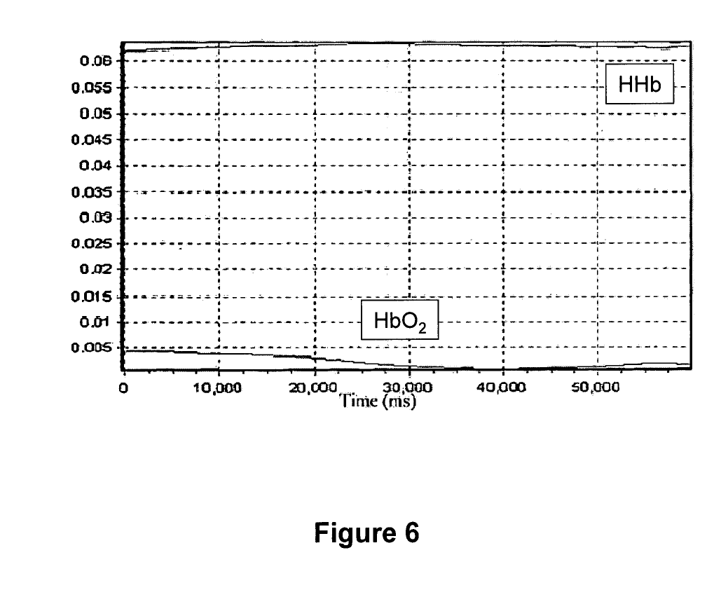

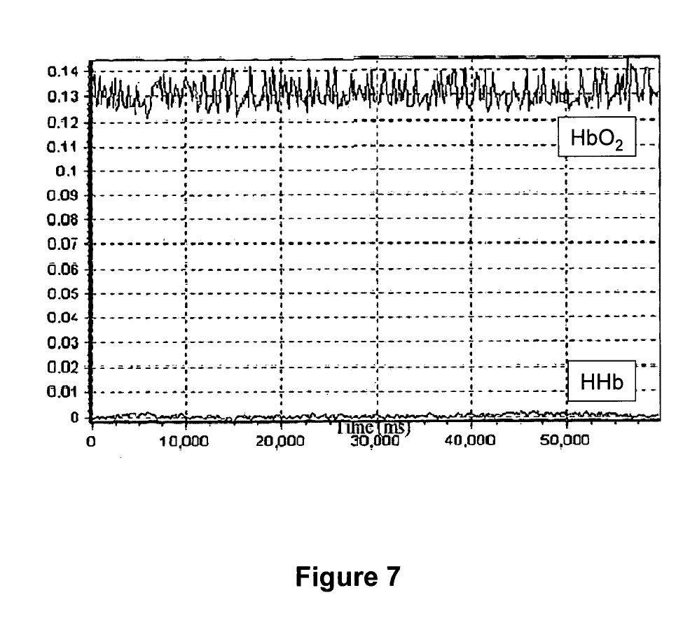

FIGS. 6-7 show HbO₂ and HHb components in a vein and artery, respectively. These plots are obtained by measuring intensity of a wavelength at about 760 nm (HHb) and 800 nm (HbO₂). As expected, the artery contains a higher concentration of HbO₂ (FIG. 7) and the vein contains a higher concentration of HHb (FIG. 6). FIG. 8 shows real-time spectral detection of blood clot formation. Thirteen absorption spectra are shown over a time ranging from about immediately after clamping to about 30 minutes post-clamping. As known in the art, blood clots can develop in no-flow conditions. This is seen in FIG. 8 by, for example, the change in absorbance spectrum between 650 nm-1000 nm. In particular, the spectrum at time t=20 minutes in FIG. 8 is similar to the spectrum of the in vitro clotted blood shown in FIGS. 4-5. Notice that the blood clot spectrum increases with time since clamping. Blood clot formation inside the vessel is confirmed by dissecting and visually inspecting the inside of the blood vessel at the end of the experiment. The forceps-like support with fiber optic attachments can be designed to complement specific surgical procedures (e.g., by-pass surgery, stent insertion, angioplasty or neurosurgery) and related surgical techniques.

The optical microprobe is used to locate, identify, localize and assess the extent of blood clots in the vasculature. Information about blood clots is particularly important for neurosurgery, but it is also useful in other surgeries. This optical method relies on the transmission of near infrared light in tissue. White light (all wavelengths-colors) is generated by a lamp or other suitable source and is delivered to the exposed vessel by a flexible fiber optic strand attached to a holder, such as a forceps-like support. Any mechanism that facilitates easy movement of the microprobe from position to position can be employed. After this light passes through the vessel, a second fiber optic strand (also connected to the holder) collects the transmitted light and delivers it to a spectrometer for spectral analysis (intensity vs. wavelength). Since tissue primarily transmits near infrared light, the spectrum is generally examined in the 600 nm to 1200 nm region. The dominant and characteristic tissue components absorbing light in this spectral window include hemoglobin (oxy- and deoxy-forms), water, fat (lipids) and a variety of minor miscellaneous compounds.

Software can be employed to facilitate the determination of the fractional spectral contributions of each component by spectral decomposition of the spectrum collected by the spectrometer based on a weighting procedure. In addition to absorption, light interacting and passing through tissue is scattered in a wavelength-dependent manner. Accordingly, the spectrum collected by the collecting fiber optic strand and subsequently analyzed is the light that is not absorbed or scattered. We have determined that the measured spectrum of tissue or blood flowing (e.g., not-clotted blood) in a vessel differs from that of a blood clot. In other words, the clot has a signature or characteristic spectrum. This is seen in FIGS. 5 and 8, where the blood spectrum and blood clot spectrum differs based on different contributions of HHb and HbO₂ toward the measured absorption spectrum. By tracing the microprobe along a vessel, and simultaneously measuring the spectrum, one can quickly identify if that region of the vessel contains a blood clot or not.

The optical microprobe of the present invention incorporates a number of established methods, such as absorption spectroscopy. Wavelength resolution of the spectrum by a spectrometer is also a well-established technique. Recognition and understanding that a blood clot spectrum differs from that of the surrounding medium and that this difference can be used to localize a clot efficiently and with good spatial resolution is not, however, understood in the art. Algorithms and experimental methods can resolve the measured spectrum into its major (additive) component parts (e.g., HHb, HbO₂, blood clot). Understanding and recognizing that blood clots generate unique absorption spectrum that can be differentiated from related HHb and HbO₂ absorption spectra, permits incorporation of a variety of technologies, such as absorption spectroscopy, fiber optic light delivery and transmission into a functional device suitable for detecting and localizing blood clots. The device and methods presented herein can be used to detect blood clots that often from during common vascular surgical procedures.

In the description of the invention, numerous specific details of the devices, device components and methods of the present invention are set forth in order to provide a thorough explanation of the precise nature of the invention. It will be apparent, however, to those of skill in the art that the invention can be practiced without these specific details.

Whenever a range is given in the specification, for example, a wavelength range, a size range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents, published patent applications, and publications mentioned in the specification are indicative of the levels of skill of those skilled in the art to which the invention pertains. References cited herein are incorporated by reference in their entirety to indicate the state of the art as of their publication or filing date and it is intended that this information can be employed herein, if needed, to exclude specific embodiments that are in the prior art.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein may be practiced in the absence of any element or elements, limitation or limitations which is not specifically disclosed herein.

One of ordinary skill in the art will appreciate that starting materials, materials, reagents, synthetic methods, purification methods, analytical methods, assay methods, and methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

Adams H, Adams R, and Del Zoppo G (2005). “Guidelines for the early management of patients with ischemic stroke: 2005 guidelines update”. A scientific statement from the Stroke Council of the American Heart Association/American Stroke Association. Stroke 36: 916-923.
Andre Roggan, Moritz Friebel, Klaus Dorschel (1999): “Optical properties of circulating human blood in the wavelength range 400-2500 nm”. Journal of biomedical Optics 4 (1): 36-46.
Andreas Raabe, M D, PhD: Jurgen Beck, M D (2003). “Near infrared Indocyanine green video angiography: A new method for Intraoperative Assessment of vascular flow”. Neurosurgery 52:132-139.
Annika M. K. Enejder, Johannes Swartling (2003): “Influence of cell shape and aggregate formation on the optical properties of flowing whole blood”. Applied optics 42(7): 1384-1393.
Charbel F T, Hoffman W E, Misra M, Ostergren L (1998): “Ultrasonic perivascular flow probe: technique and application in Neurosurgery”. Neurological Research 20: 439-442:
Charbel F T, MD: Guido Meglio, M D (2005). “Superficial temporal artery-to-middle cerebral artery Bypass”. Neurosurgery 56 (ONS Suppl. 1) 186-190.
Charbel F T, Hoffman W E, Misra M, Hannigan K, Ausman J I (1998). “Role of a perivascular ultrasonic micro flow probe in aneurysm surgery”. Neurologia Medico-Chirurgica 38: 35-38.
F. Neudel, S. Takatani (2004): “Effect of Hemolysis on oxygen and hematocrit measurements by near infrared reflectance spectroscopy”. Medical Engineering Physics 24: 301-307.
Frank A. Greco, M D, PhD (2004). “Reflectance Spectroscopy of Clotting Blood. A description of the Time-Dependent Behavior”. Arch Pathol. Lab Med. 128: 173-180.
Jarold C. Finlay, Thomas Foster (2004): “Effect of pigment packaging on diffuse reflectance spectroscopy of samples containing red blood cells”. Optical Society of America 29(9): 965-967.
Werner Jaross, Volker Neumeister (1999): “Determination of Cholesterol in arteriosclerotic plaques using near infrared diffuse reflection spectroscopy”. Arteriosclerosis 147: 327-337.
Wiendelt Steenberg, Roy Kolkman (1999). “Light-scattering properties of undiluted human blood subjected to simple shear”. J. Opt Soc Am. 16(12): 2959-2967.
U.S. Pat. No. 6,104,939: “Method and apparatus for reflected imaging analysis”; U.S. Pat. No. 6,178,346 “Infrared endoscopic Imaging in a liquid with suspended particles: Method and apparatus”; U.S. Pat. No. 5,282,467 “Non-Invasive method for detecting deep venous thrombosis in the human body”; U.S. Pat. No. 6,775,565 “Imaging apparatus for displaying concentration ratios”; U.S. Pat. No. 5,792,051 “Optical probe for Non-invasive Monitoring of Neural Activity”
US Pub Nos: 2003/0236458 “Spectroscopic System and Methods for detecting tissue properties”; 2004/0111016A1 “Method and apparatus for detection of vulnerable Atherosclerotic plaque”

We claim:

1. An optical microprobe for non-invasively detecting blood clots in a blood vessel by transmission spectroscopy, said optical microprobe comprising:
a. an optical source for generating electromagnetic radiation having a wavelength range corresponding to the wavelength of near infrared light and capable of being absorbed by a blood clot;
b. a first fiber optic strand capable of transmitting the electromagnetic radiation generated by the optical source, the strand having a proximal end and a distal end, wherein the proximal end is optically connected to the optical source, and the distal end is capable of illuminating the blood vessel with the electromagnetic radiation;
c. a second fiber optic strand capable of transmitting the electromagnetic radiation generated by the optical source, the strand having a proximal end and a distal end, wherein the distal end is capable of collecting the electromagnetic radiation that has illuminated and passed through the blood vessel; and
d. a holder having a first holding arm connected to the first fiber optic strand distal end and a second holding arm connected to the second fiber optic strand distal end, wherein the holder stably positions the distal portions of the first and second fiber optic strands in a diametrically opposed configuration and separated by a separation distance.

2. The optical microprobe of claim 1 wherein each of the holding arms has a bottom end, the optical microprobe further comprising:
a. a first holding tip connected to the first holding arm bottom end; and
b. a second holding tip connected to the second holding arm bottom,
wherein, the first holding tip is connected to the distal end of the first fiber optic strand, and the second holding tip is connected to the distal end of the second fiber optic strand.

3. The optical microprobe of claim 2, wherein the holding tip further comprises an orifice, and the distal end of the fiber optic strand is disposed within the orifice.

4. The optical microprobe of claim 1, further comprising means for selecting the separation distance.

5. The optical microprobe of claim 4, wherein the separation distance is selected from a range of about 0.5 mm to about 2 cm.

6. The optical microprobe of claim 1, further comprising a micromanipulator connected to the holder for controllable positioning of the distal ends of the fiber optic strands.

7. The optical microprobe of claim 1, further comprising an optical detector optically connected to the second fiber optic strand proximal end for detecting the electromagnetic radiation collected by the second fiber optic strand distal end.

8. The optical microprobe of claim 7, further comprising an analyzer for determining the intensity of electromagnetic radiation at a wavelength or wavelength range corresponding to the wavelength absorbed by a blood clot.

9. The optical microprobe of claim 8, wherein the wavelength range is selected from between about 600 nm and 1000 nm.

10. The optical microprobe of claim 8, wherein the analyzer determines the intensity of electromagnetic radiation having a wavelength range of 660 nm to 990 nm.

11. The optical microprobe of claim 8, wherein the analyzer determines a spectral contribution due to absorption of the electromagnetic radiation by a spectral component, the spectral component selected from the group consisting of oxyhemoglobin and deoxyhemoglobin.

12. The optical microprobe of claim 1, further comprising a microdrive operably connected to the holder to provide blood clot location detection along at least an axial portion of the blood vessel.

13. The optical microprobe of claim 1, wherein the first and second optic fiber strand distal ends are capable of physical contact with the blood vessel outer wall.

14. The optical microprobe of claim 13, wherein the blood vessel has a diameter selected from the range of 0.5 mm to 2 cm.

15. The optical microprobe of claim 1, wherein the optical source generates electromagnetic radiation substantially restricted to the near infrared portion of the electromagnetic spectrum.

16. A method for detecting clots in a blood vessel comprising:
a. providing a first optical fiber having one end in optical contact with the outer surface of the blood vessel and the other end in optical contact with an optical source;
b. providing a second optical fiber in optical contact with the outer surface of the blood vessel, wherein the first and second optical fibers are positioned in a diametrically-opposed configuration;
c. illuminating the blood vessel with electromagnetic radiation produced by the optical source, wherein the electromagnetic radiation comprises a wavelength that is capable of being absorbed by a blood clot within the blood vessel and at least a portion of the illuminating radiation passes through the blood vessel;
d. collecting with the second optical fiber at least a portion of the electromagnetic radiation that has passed through the blood vessel,
e. detecting from the collected electromagnetic radiation, a radiation spectrum having a wavelength between about 600 nm and 1000 nm, wherein the spectrum is sensitive to blood clots; and
f. analyzing the detected radiation to determine the presence or absence of a blood clot.

17. The method of claim 16, wherein the analyzing step further comprises determining the spectrum over a wavelength range of between 650 nm and 980 nm.

18. The method of claim 16, wherein the analyzer compares the spectrum of the detected radiation with a standard blood clot spectrum, wherein the standard blood clot spectrum is obtained from an in vitro blood clot.

19. The method of claim 16, wherein the analyzing step further comprises determining a spectral contribution of a one or more spectral components selected from the group consisting of oxyhemoglobin and deoxyhemoglobin.

20. The method of claim 19, wherein the blood clot is detected by measuring a rate of change or a total change of the spectral contribution of the one or more spectral components and comparing the measured rate of change or total change to a baseline value.

21. The method of claim 16 further comprising detecting from the spectrum one or more blood parameters selected from oxyhemoglobin, deoxyhemoglobin and total hemoglobin.

22. The method of claim 16 wherein the blood vessel has a length, said method further comprising:
a. moving the optical fibers along at least a portion of the blood vessel length; and
b. obtaining a radiation spectrum along at least a portion of the blood vessel length, thereby determining the location of the blood clot.

23. The method of claim 16, wherein the method is carried out before or during a surgical procedure.

24. The method of claim 23, wherein the surgical procedure is selected from the group consisting of:
a. brain aneurysm repair;
b. ischemic stroke repair;
c. arteriovenous malformation repair;
d. peripheral artery disease repair;
e. reconstructive plastic surgery;
f. moyamoya disease repair;
g. vascular stent insertion;
h. vascular stent removal; and
i. bypass anastomoses for revascularization.

25. A method of determining the position of a blood clot if present in a blood vessel comprising:
a. providing the optical microprobe of claim 1;
b. positioning the optical microprobe so that the blood vessel is between the distal ends of the first and second fiber optic strands;
c. illuminating the blood vessel with electromagnetic radiation;
d. collecting the electromagnetic radiation that has passed through the blood vessel;
e. analyzing the collected electromagnetic radiation to determine whether a blood clot is present; and
f. repeating steps b-e at a different axial blood vessel location, thereby determining the position of the blood clot if present within the blood vessel.
